SEQUENCE LISTING (1 sequence)

SEQ ID NO: 1 - DNA, 1017 bp, redspotted grouper
atggtacgcaaaggtgagaagaaattggcaaaacccgcgaccactaaggccgcgaatccgcaaccccgccgacgtgctaacaatcgtcggcgtagtaatcgcactgacgcacctgtgtctaaggcctcgactgtaactggatttggacgtgggaccaatgacgtccatctctcaggtatgtcgagagtctcccaggccgtcctcccagccgggacaggaacagacggatacgttgttgttgatgcaaccatcgtccccgacctcctgccacgactgggacacgctgctagaatcttccagcgatacgctgttgaaacactggagtttgaaattcagccaatgtgccccgcaaacacgggcggtggttacgttgctggcttcctgcctgatccaactgacaacgatcacaccttcgacgcgcttcaagcaactcgtggtgcagtcgttgccaaatggtgggaaagcagaacagtccgacctcagtacacccgcacgctcctctggacctcgtcgggaaaggagcagcgtctcacgtcacctggtcggctgatactcctgtgtgtcggcaacaacactgatgtggtcaacgtgtcagtgctgtgtcgctggagtgttcgactgagcgttccatctcttgagacacctgaagagaccaccgctcccatcatgacacaaggttccctgtacaacgattccctttccacaaatgacttcaagtccatcctcctaggatccacaccactggacattgcccctgatggagcagtcttccagctggaccgtccgctgtccattgactacagccttggaactggagatgttgaccgtgctgtttactggcacctcaagaagtttgctggaaatgctggcacacctgcaggctggtttcgctggggcatctgggacaacttcaacaagacgttcacagatggcgttgcctactactctgatgagcagccccgtcaaatcctgctgcctgttggcactgtctgcaccagggttgactcggaaaactaa

(19) United States
(12) Patent Application Publication
(10) Pub. No.: US 2008/0070289 A1
(43) Pub. Date: Mar. 20, 2008

(54) PROCESS FOR STORING ENRICHED NEMATODES

(75) Inventor: Shalom Zemach, Kfar Yona (IL)

Correspondence Address: DR. MARK M. FRIEDMAN;C/O BILL POLKINGHORN - DISCOVERY DISPATCH, 9003 FLORIN WAY, UPPER MARLBORO, MD, 20772, US

(73) Assignee: Fish Biotech Ltd., Kfar Yona (IL)

(21) Appl. No.: 11/841,008

(22) Filed: Aug. 20, 2007

Related U.S. Application Data
(63) Continuation-in-part of application No. 10/466,610, filed on Jul. 18, 2003, now abandoned.

(51) Int. Cl. C12N 1/04

(52) U.S. Cl. 435/260

(57) ABSTRACT

A process for preserving and storing nematodes having an increased feeding value for later use as needed in the feeding of aquaculture organisms. In this process, nematodes are enriched with an additive such as essential fatty acids, vaccines, hormones, immunostimulants, attractants, nutrients and pigments. Liposomes can serve as a medium for feeding the additives to the nematodes. The enriched nematodes are then desiccated by inducing a quiescent anhydrobiosis (dehydration) and stored for off-the-shell use as needed. When required for feeding, the desiccated enriched nematodes are rehydrated and fed to the aquaculture organisms.

This is a Continuation-in-Part of U.S. patent application Ser. No. 10/466,610 filed Jul. 18, 2003, which is national phase of PCT/IL2002/00434 filed Jun. 4, 2002 which claims priority from U.S. Ser. No. 09/893,875, patented.

### FIELD AND BACKGROUND OF THE INVENTION

The present invention relates to aquaculture and, more particularly, to a method for “off-the-shelf” storage of enriched free-living nematodes used in feeding aquaculture organisms such as fish and shrimp larvae.

A major bottleneck to the successful growing of marine fish with commercial potential is the rearing of the early developmental stages or larvae. During this time, mortality can range from about 60-100%, frequently due to insufficient or poor nutrition. For instance, hatcheries depend on the provision of live food or zooplankton to the larvae such as rotifers (Brachionus plicatilis or other Brachianus sp.) and brine shrimp (Artemia sp.). These zooplankters do not represent the natural diet but are relatively easy to grow in large quantities and are readily accepted by the larvae. On the other hand, the culture of these zooplankters requires a considerable investment in infrastructure (tanks, water and air pumps, water treatment) as well as energy and manpower. Moreover, the live food cannot be stored or is deficient in specific long chain polyunsaturated fatty acids (PUFA), such as docosahexaenoic acid (DHA) and eicosapentaenoic acid (EPA) which are necessary in the diet for rapidly growing larvae which can demonstrate daily a relative growth rate (RGR) of 25-50% (Koven, W., Tandler, A., Kissil, G. Wm., Sklan, D. 1992 The Importance of n-3 highly unsaturated fatty acids for growth in larval Sparus aurata and their effect on survival, lipid composition and size distribution. Aquaculture 104, 91-104). This is also true with arachidonic acid (ArA) which has recently been recognized as playing a central role in increasing stress resistance (Koven, W., Barr, Y., Lutzky, S., Ben-Atia. I., Weiss, R., Harel, M., Behrens, P., Tandler, A. 2001. The effect of arachidonic acid (20:4n-6) on growth, survival and resistance to handling stress in gilthead seabream (Sparus aurata) larvae. Aquaculture 193, 107-122). Consequently, rotifers and Artemia must be enriched with these PUFA, by feeding them commercial DHA and EPA preparations, before they are offered to the fish larvae.

Live food culture systems are frequently plagued by an inconsistent supply of zooplankters that have variable nutritional content and are susceptible to sudden population crashes. So for instance an interruption in the supply and/or an inconsistency in feed quality of food fed to the larvae can severely reduce their growth rate, extending the residence time in the hatchery or resulting in smaller, less robust larvae being transferred to the nursery, resulting in reduced growth and survival. In addition, poor feeding can cause stress, resulting in a decreased resistance to disease. In recent years, primarily due to over exploitation, there has been a rapid decline in the world wide availability of Artemia cysts resulting in fluctuating prices and reduced cyst quality compounding the problems of providing these zooplankters.

A clear advantage in the use of free-living (non-parasitic) nematodes as a feed is in that the nematodes can be stored in a dormant-desiccated state, after enrichment. They can be encapsulated and revived at a later stage for feeding to larvae. This “off the shelf convenience” provides a dependable and nutritionally consistent food supply to the larvae that can be provided, with less investment, than other conventional live food species.

Essential fatty acids for marine larvae such as docosahexaenoic acid (22:6n-3), eicosapentaenoic acid (20:5n-3) and arachidonic acid (20:4n-6) can be provided to the nematodes through various oil emulsions. Previous studies have shown that nematodes readily filter the micelles of oil emulsions. Recent studies have shown that the use of liposomes to feed nematodes is a promising approach to widen the range of feeding additives that could be used for enrichment. Liposomes are small (0.025-1 .mu.m) lipid vesicles consisting of an aqueous volume surrounded by a bi-lamellar phospholipid membrane. It is relatively easy to incorporate water-soluble vitamins, minerals, proteins and amino acids into the aqueous volume and/or lipid-soluble nutrients such as lipids, vitamins and pigments into the liposome's phospholipid membrane (Koven, W., Barr, Y., Iladas, E., Ben-Atia, I., Chen, Y., Weiss, R., Tandler, A. 1999. The potential of liposomes as a nutrient supplement in first-feeding marine fish larvae. Aquaculture Nutrition 5, 251-256).

A recent study showed that liposomes could be used to enrich Artemia nauplii with the free amino acid methionine (Tonheim, S. K., Koven, B, Ronnestad, I. 2000. Enrichment of Artemia with free methionine. Aquaculture 190, 223-235). This zooplankter is generally deficient in this amino acid and its enrichment may contribute to more efficient protein synthesis. This approach has recently been expanded to include the enrichment of nematodes with this and other free amino acids as well as free fatty acids, which stimulate digestive hormones in the larvae such as cholecystokinin (CCK). CCK is a major factor in the release of pancreatic enzymes resulting in enhanced digestion and assimilation of dietary nutrients. In addition, liposomes fed to nematodes containing immunostimulants, vaccines and other pharmaceuticals may stimulate disease and stress resistance in the larvae resulting in improved larval and juvenile fish quality.

U.S. Pat. No. 5,183,950 to Popiel et al teaches a method for commercial storage and shipment of entemogenous (parasitic to insects) nematodes. It relates to methods to desiccate, package, store, and ship insect parasitic nematodes in both large and small quantities while maintaining their viability and pathogenicity to insects. Popiel's method does not relate to free-living (non-parasitic) nematodes and does not mention enriching the nematodes before storage.

U.S. Pat. No. 5,042,427 to Bedding describes a method of storing and transporting enteropathogenic nematodes by using clay to dry the nematodes, which are revived when dispersed in water. Bedding's method does not mention enriching the nematodes before storage.

Desiccation of nematodes is also taught by Solomon et al. (Solomon A., Papema I., Glazer I. 1999 Desiccation survival of the entomopathogenic nematode Steinernema feltiae: induction of anhydrobiosis Nematology 1 (1), 61-68) as well as by Perry (Perry R. 1999 Desiccation survival of parasitic nematodes. Parasitology 119,S19-S30). Here again there is no mention of enriching the nematodes before desiccation.

WO Pat. No. 95/18527 to Agricultural Genetics Company LTD teaches enriching nematodes with various additives such as various oils and pigments for use as live feed for larvae. This method illustrates the advantages of feeding enriched nematodes to larvae but there is no mention of long-term storage of the nematodes.

The free living nematode Caenorhabditis elegans (C. elegans) has been used as a model genetic organism and has been the subject of intensive molecular and genetic analysis (Jorgensen E M., and Mango S E. 2002, The art and design of genetic screens: Caenorhabditis elegans. Nature Reviews Genetics 3: 356-369; The C. elegans Sequencing Consortium. 1998 Genome sequence of the nematode C. elegans: a platform for investigating biology. Science 282: 2012-2018; Riddle D. L., Blumenthal, T., Meyer, B J., and Priess, J R. C. elegans II. Cold Spring Harbor Laboratory Press, New York, 1997.) There has not been however any mention of enrichment, preservation or storage of C. elegans for off-the-shelf use in aquaculture.

There is thus a widely recognized need for, and it would be highly advantageous to have, a process for preserving and storing free-living nematodes having an increased feeding value devoid of the above limitations.

### SUMMARY OF THE INVENTION

According to one aspect of the present invention there is provided a process for preserving free-living nematodes having an increased feeding value. The process comprises the steps of enriching free-living nematodes with aquaculture feed additives and desiccating enriched nematodes.

According to another aspect of the present invention there is provided an improved method of aquaculture. The method comprises the steps of enriching nematodes with aquaculture feed additives, desiccating enriched nematodes, storing the desiccated enriched nematodes, rehydrating and reviving the desiccated enriched nematodes; and feeding rehydrated enriched nematodes to an organism being grown in the aquaculture.

According to another aspect of the present invention there is provided an improved method of aquaculture. The method comprises the steps of desiccating nematodes, storing the desiccated nematodes, rehydrating and reviving the desiccated nematodes, enriching nematodes with aquaculture feed additives and feeding rehydrated enriched nematodes to an organism being grown in the aquaculture.

According to another aspect of the present invention there is provided a process for maintaining nematodes having an increased feeding value, for use in aquaculture. The process includes the steps of enriching nematodes with aquaculture feed additives and preserving enriched nematodes.

According to still another aspect of the present invention, there is provided an improved method of aquaculture. The method includes the steps of enriching nematodes with aquaculture feed additives; preserving enriched nematodes; storing desiccated enriched nematodes; rehydrating and reviving said desiccated enriched nematodes; and feeding rehydrated enriched nematodes to an organism being grown in the aquaculture.

According to yet another aspect of the present invention, there is provided an improved method of aquaculture. The method includes the steps of preserving nematodes; storing said desiccated nematodes; rehydrating and reviving said desiccated nematodes; enriching nematodes with feed additives; and feeding rehydrated enriched nematodes to an organism being grown in the aquaculture.

According to still another aspect of the present invention, there is provided a process for maintaining free-living nematodes having an increased feeding value, for use in aquaculture, comprising the steps of (a) enriching nematodes with aquaculture feed additives; and (b) preserving enriched nematodes, wherein the step of preserving enriched nematodes includes lowering the temperature of the enriched nematodes to significantly reduce the metabolism of the enriched nematodes to the point where the metabolism of the enriched nematodes is slowed down but not suppressed to the point to dormancy, so as to maintain the nutritional value of the nematodes for feeding in aquaculture.

According to a further aspect of the present invention, there is provided a process for maintaining free-living nematodes having an increased feeding value, for use in aquaculture, comprising the steps of (a) enriching nematodes with aquaculture feed additives; and (b) preserving enriched nematodes, wherein the step of enriching nematodes includes genetic manipulation of the nematode. The process may further comprise the steps of (c) culturing the nematodes and optionally (d) storing the preserved enriched nematodes.

According to yet another aspect of the present invention, there is provided an improved method of aquaculture comprising the steps of (a) preserving free-living nematodes; (b) storing said preserved nematodes; (c) rehydrating and reviving said stored preserved nematodes; (d) enriching nematodes with feed additives; and (e) feeding rehydrated enriched nematodes to an organism being grown in the aquaculture, wherein the step of preserving the nematodes includes lowering the temperature of the nematodes to significantly reduce the metabolism of the nematodes to the point where the metabolism of the nematodes is slowed down but not suppressed to the point to dormancy, so as to maintain the nutritional value of the nematodes for feeding in aquaculture.

According to further features in preferred embodiments of the invention described below, the process further comprises the step of culturing nematodes.

According to still further features in the described preferred embodiments, the process further comprises the step of storing the desiccated nematodes.

According to still further features in the described preferred embodiments, the nematodes are free-living nematodes.

According to still further features in the described preferred embodiments, the free-living nematodes are of the species Panagrellus.

According to still further features in the described preferred embodiments, the free-living nematodes are of the species Caenorhabditis elegans.

According to still further features in the described preferred embodiments, the step of enriching nematodes includes providing at least one enriching additive selected from the group consisting of essential fatty acids, vaccines, hormones, immunostimulants, attractants, nutrients and pigments.

According to still further features in the described preferred embodiments, the step of desiccating the nematodes is performed by inducing one item selected out of the group consisting of quiescent anhydrobiosis and osmobiosis.

According to still further features in the described preferred embodiments, the step of enriching nematodes includes genetic manipulation of the nematode.

According to still farther features in the described preferred embodiments, the step of enriching is affected by feeding the nematodes with liposomes containing enriching additives.

According to still further features in the described preferred embodiments, the step of preserving of the nematodes includes dessicating the nematodes.

According to still further features in the described preferred embodiments, the step of preserving of nematodes includes reducing significantly metabolism of the nematodes by lowering a temperature of the nematodes.

Among the advantages of off-the shelf convenience are:

a) The ability to store enriched nematodes ensures constant food supply for the larvae. Due to the rapid daily larval growth rate (RGR of 25-50%), the interruption of food supply, as a result of equipment failure, crashes in rotifer and algal cultures and lower hatching rates in poor quality Artemia cysts, can translate into less than optimal larval growth and survival.

b) As batches of enriched nematodes can be tested for nutrient content before storage, consistent nutritional quality can be ensured before feeding larvae.

c) The ability to produce a product for storage, means that sufficient reserves can be built up. This is advantageous otherwise further investment in infrastructure, labor and energy as compared to any other traditional food supply method will be necessary.

d) The ability to store enriched nematodes is a basis for a commercial product that could compete with Artemia cysts, which is a resource plagued by dwindling supply, variable quality, occasional contamination with pesticides fluctuating high prices, and is a non-sustainable resource.

e) Newly hatched Artemia's body composition can reflect only natural processes in the environment in which the Artemia broodstock dwelled. Cyst laying, being a natural process, lends NO control over quality of the hatched nauplii, in terms of body composition. On the other hand nematodes, which address the same size larvae, can be enriched prior to their encapsulation for long-term storage.

The present invention successfully addresses the shortcomings of the presently known configurations by providing a process for preserving and storing nematodes having an increased nutritional value for later use as needed in the feeding of aquaculture organisms.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art of how the several forms of the invention may be embodied in practice.

In the Drawings:

FIG. 1 is a flow chart of an improved method of aquaculture.

FIG. 2 is an open reading frame encoding for the redspotted grouper nervous necrosis virus (VNN) coat protein (GenBank accession number AY744705)

FIG. 3 is a plasmid vector for expression in nematodes harboring an active promoter (P), a nucleotide sequence coding for a green fluorescent protein (GFP) translationaly fused to the coding sequence of target gene (e.g. VNN coat protein) and a nucleotide sequence coding for termination of translation (TS).

### DESCRIPTION OF THE PREFERRED EMBODIMENTS

The present invention is of a method of aquaculture, which can be used for feeding larvae. Specifically, the present invention can be used to preserve and store enriched desiccated nematodes so that they may be taken “off-the-shelf”, be rehydrated and revived for use as needed. This method omits the need for live aquaculture feed such as Artemia, rotifers, copepods etc.

The principles and operation of a method of aquaculture, according to the present invention, may be better understood with reference to the drawing and accompanying descriptions.

Before explaining at least one embodiment of the invention in detail, it is to be understood that the invention is not limited in its application to the details of construction and the arrangement of the components set forth in the following description or illustrated in the drawing. The invention is capable of other embodiments or of being practiced or carried out in various ways. Also, it is to be understood that the phraseology and terminology employed herein is for the purpose of description and should not be regarded as limiting.

For purposes of this specification and the accompanying claims, the term “nematode” refers to free-living nematodes (i.e. non-parasitic) nematodes, more preferably with the species Panagrellus as well as C. elegans.

For purposes of this specification and the accompanying claims, the term “desiccation” refers to removal of water from an organism by evaporation, by osmotic pressure or by any other means serving this purpose.

For purposes of this specification and the accompanying claims, the term “aquaculture” refers to the rearing and propagation of any aquatic organism.

For purposes of this specification and the accompanying claims, the term “enrich” refers to substances provided to nematodes via their diet, medium or otherwise, such as by genetic manipulation, to increase the level of the respective substance in the nematode body prior to feeding it to the target organism and may include but are not restricted to essential fatty acids, vaccines, hormones, immunostimulants, attractants, nutrients or pigments.

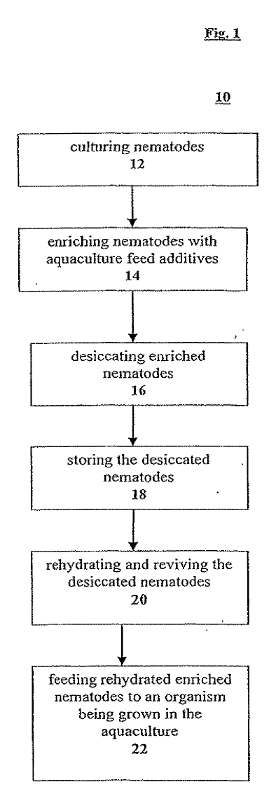

Referring now to the drawings, FIG. 1 is a flow chart illustrating a method 10 for preserving nematodes having an increased feeding value. Method 10 includes the step of culturing nematodes 12. Nematode culturing is known to those skilled in the art of nematology. Nematodes may be fed with yeasts and bacteria acting as mediators for delivering food to the nematodes. Alternately, or additionally, nematodes may be grown on an industrial scale with the use of fermentors.

According to method 10 of the present invention, nematodes are “enriched” with aquaculture feed additives 14 just prior to or alternately after harvest. The nematodes are enriched with at least one additive such as essential fatty acids, vaccines, hormones, immunostimulants, attractants, nutrients or pigments, which is added to the feed of the nematodes. A mediator that has been successfully used for feeding the additives to the nematodes is by penetrating the additives to liposomes and then feeding the liposomes to the nematodes. Essential fatty acids for marine larvae such as docosahexaenoic acid (22:6n-3), eicosapentaenoic acid (20:5n-3) and arachidonic acid (20:4n-6) can be provided to the nematodes through various oil emulsions. In addition liposomes, containing immunostimulants and vaccines, can be fed to nematodes, which may improve disease resistance in the larvae feeding on them. Moreover, nematodes can be fed liposomes containing specific free amino (FAA) and fatty acids (FFA) that stimulate digestive hormones in the larvae such as cholecystokinin (CCK). CCK is a major factor in the release of pancreatic enzymes resulting in enhanced digestion and assimilation of dietary nutrients. Other FAA can be incorporated in liposomes to stimulate appetite or to improve larval protein synthesis by giving a more balanced amino acid composition.

In alternative preferred embodiments, enrichment of nematodes with aquaculture feed additives (14) is accomplished by a different approach. In these preferred embodiments, enrichment is accomplished by genetic manipulation of the nematode genome. Such an approach is most easily facilitated using a nematode such as the well-studied nematode C. elegans. The genome of C. elegans has been fully sequenced (The C. elegans Sequencing Consortium. 1998 Genome sequence of the nematode C. elegans: a platform for investigating biology. Science 282: 2012-2018). Using molecular biological techniques various genes and gene sequences in C. elegans can be manipulated (including both regulatory elements and structural genes) to increase the expression of various nutrient compounds and components intrinsic to the species. Alternatively, new genetic sequences can be introduced into the nematode genome to enrich the nutritional content of the nematode or to produce other biopharmaceutical type compounds, including, but not limited to, essential fatty acids, vaccines, hormones, growth factors, immunostimulants, attractants, nutrients or pigments.

Introduction of genetic material can utilize various techniques for producing transgenic nematodes including, as non-limiting examples, target selected mutagenesis and manipulation and mobilization of known transposable elements (e.g., Tc1) as well as various standard techniques for gene introduction into C. elegans including microinjection (Riddle D. L., Blumenthal, T., Meyer, B J., and Priess, J R. C. elegans II. Cold Spring Harbor Laboratory Press, New York, 1997.).

Transgenic lines of C. elegans are generated by transformation of C. elegans with exogenous DNA by microinjection of vectors carrying DNA sequences into the syncitial ovaries of adult hermaphrodites. The injected DNA forms multimerized tandem arrays of microinjected DNA carried either as semi-stable extrachromosomal arrays, or integrated into a host chromosome, forming a stable integrated transgenic line. By including appropriate genetic markers with the injected DNA, one can identify animals that carry the transgene. Sometimes chromosomal integration of the introduced DNA can occur, or an existing extrachromosomal array can be integrated after irradiation of a transgenic line. See Riddle D. L., Blumenthal, T., Meyer, B J., and Priess, J R. C. elegans II. Cold Spring Harbor Laboratory Press, New York, 1997; Hashmi, S., G. Hashmi & R. Gaugler. 1995. Genetic transformation of an entomopathogenic nematode by microinjection. J. Invertebr. Pathol. 66:293-6; Methods in Cell Biology Vol 48 Caenorabhditis elegans—Modern Biological analysis of an organism, Eds Epstein and Shakes, Academic Press, 1995; Kwa M S., Veenstra, J G., Van Dijk, M., and Roos, M H. 1995 Beta-tubulin genes from the parasitic nematode Haemonchus contortus modulate drug resistance in Caenorhabditis elegans. J Mol Biol 246(4):500-10.; Fire, A. 1986. Integrative transformation of Caenorhabditis elegans. EMBO J 5:2673-2680 for example.

After feeding the nematodes with the enriching additive, the enriched nematodes are ready for feeding to the breeding larvae or are desiccated 16 for future “off-the-shelf” use. Several prior methods of desiccation are discussed above in the introduction and are known to those skilled in the art of nematology. A preferred method of desiccation is performed by inducing a quiescent anhydrobiosis or osmobiosis. Anhydrobiosis/osmobiosis or dehydration of the nematodes is optimally a quiescent anhydrobiosis. Quiescence is a spontaneous reversible response to unpredictable unfavorable environmental conditions and release from quiescence occurs when favorable conditions return. The desiccated and enriched nematodes are then stored 18 and are available in a dehydrated state for use as needed at a future date.

In alternative preferred embodiments of the method of the present invention, in place of dessication (16), other techniques for preserving enriched nematodes can be employed. For example, after enrichment, the enriched nematodes may be preserved for future “off-the-shelf” use by lowering the temperature of the enriched nematodes to significantly reduce the metabolism of the enriched nematodes, to the point where the metabolism of the enriched nematodes is slowed down but not suppressed to the point of dormancy, so as to maintain the nutritional value of the nematodes for feeding in aquaculture. The temperature of the enriched nematodes is lowered to reduce the metabolism of the enriched nematodes, but not to the point of dormancy where metabolism is totally suppressed. The temperatures are lowered sufficiently to achieve a general reduction in the rate of metabolism of the enriched nematodes, but not to achieve dormancy, which is a condition of biological rest or inactivity characterized by cessation of growth or development and the suspension of many metabolic processes. Prior to use, the nematodes can be restored and revived by raising the temperature of the preserved nematodes to a normal temperature.

FIG. 1 further illustrates an improved method 10 of aquaculture. The method includes the step of culturing nematodes 12. Nematode culturing is known to those skilled in the art of nematology. Nematodes may be fed with yeasts and bacteria acting as mediators for delivering food to the nematodes. Alternately, or additionally, nematodes may be grown on an industrial scale with the use of fermentors.

According to method 10 of the present invention, after a period of time, before harvesting or alternately after harvesting, the nematodes are enriched with aquaculture feed additives 14. A novel means of incorporating feed additives into the nematodes is by using a mediator. A mediator which has been successfully used for feeding the feed additives to the nematodes are liposomes which contain feed additives. Alternately, or additionally, other mediators can be utilized for this purpose.

After feeding the nematodes with the enriching additive, the enriched nematodes are ready for feeding to the breeding larvae or are desiccated 16 for future use. Several prior methods of desiccation are discussed above in the introduction and are known to those skilled in the art of nematology. The desiccated enriched nematodes are then stored 18 and are available in a dehydrated state for off-the shelf use at a future date.

Before use, the desiccated enriched nematodes are rehydrated and revived 20. Following rehydration, the rehydrated enriched nematodes are fed to an organism being grown in aquaculture 22.

In an alternative embodiment of the present invention nematodes which have been cultured are desiccated and then stored. Prior to use the desiccated nematodes are rehydrated and revived and then the nematodes are enriched with feed additives and then fed to an organism being grown in the agriculture.

In alternative preferred embodiments of the present invention, enrichment of nematodes with aquaculture feed additives (14) prior to preservation is accomplished by genetic manipulation of the nematode genome, as described hereinabove and in Example 1 below. In further alternative embodiments, in place of dessication, the enriched nematodes are preserved by lowering the temperature of the enriched nematodes to reduce significantly the metabolism of the enriched nematodes to the point where the metabolism of the enriched nematodes is slowed down but not suppressed to the point of dormancy, so as to maintain the nutritional value of the nematodes for feeding in aquaculture. Prior to use, the nematodes can be restored and revived by gradually raising the temperature of the preserved nematodes to a normal temperature, and then fed to an organism being grown in the aquaculture. The gradual arousal from a reduced temperature state (if not too low) is in contrast to the abrupt arousal which occurs upon arousal from a dormant stage.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); Riddle D. L., Blumenthal, T., Meyer, B J., and Priess, J R. C. elegans II. Cold Spring Harbor Laboratory Press, New York, 1997; Hashmi, S., G. Hashmi & R. Gaugler. 1995. Genetic transformation of an entomopathogenic nematode by microinjection. J. Invertebr. Pathol. 66:293-6; Methods in Cell Biology Vol 48 Caenorabhditis elegans—Modern Biological analysis of an organism, Eds Epstein and Shakes, Academic Press, 1995; Kwa M S., Veenstra, J G., Van Dijk, M., and Roos, M H. 1995 Beta-tubulin genes from the parasitic nematode Haemonchus contortus modulate drug resistance in Caenorhabditis elegans. J Mol Biol 246(4):500-10.; Fire, A. 1986. Integrative transformation of Caenorhabditis elegans. EMBO J. 5:2673-2680; Jorgensen E M., and Mango S E. 2002 The art and design of genetic screens: Caenorhabditis elegans. Nature Reviews Genetics 3: 356-369; and The C. elegans Sequencing Consortium. 1998 Genome sequence of the nematode C. elegans: a platform for investigating biology. Science 282: 2012-2018; all of which are incorporated by reference as if fully set forth herein. Other general references are provided throughout this document. All the information contained therein is incorporated herein by reference.

### EXAMPLE 1

Transgenic lines of nematodes can be used as a platform that expresses heterologous vaccine(s), and induces protective immunity following oral consumption by the fish larvae.

Many viral diseases, including the viral nervous necrosis (VNN), cause high mortality among various species of marine fish larvae, resulting in serious economic losses for the aquaculture industry. To overcome these problems, effective vaccines must be administered at the early larval stage prior to the occurrence of viral infection. Nevertheless, due to their small size and high sensitivity to stress, an oral vaccination appears to be a better approach for larvae immunization than injection.

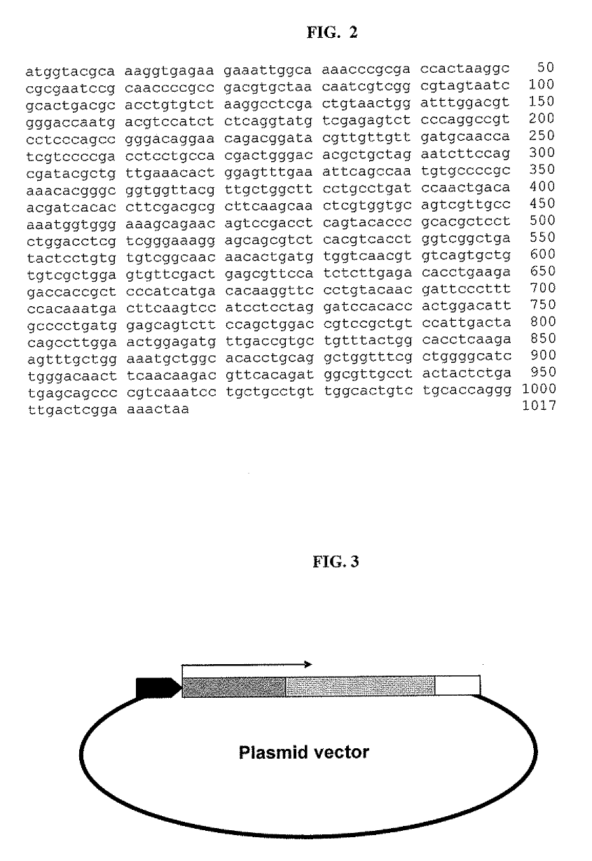

Hence, to generate, for instance, transgenic nematodes that function as a VNN-vaccine, the respective DNA sequence encoding for VNN coat protein (FIG. 2; GenBank accession number: AY744705), can be subcloned into a chosen plasmid vector (from A. Fire Labs, Carnegie Institution of Washington, Department of Embryology, Baltimore, Md., USA), downstream of an active promoter (inducible or constitutive), followed by signals to allow processing and expression of the resultant transcripts. Alternatively, the green fluorescent protein (GFP) coding region can be fused to either 5′ or 3′ ends of the target gene as illustrated in FIG. 3. Commonly, such translational fusions retain the function of the target gene product while tagging it with the fluorescent properties of GFP. Thus, in addition to its role in screening transgenic lines of nematodes, the GFP can be detected by the larvae and serve as a beacon attracting larvae to the nematode prey resulting in improved feeding and antigen uptake. Then the chimeric plasmids can be used to transform nematodes by microinjection into the central core of cytoplasm in each arm of the hermaphrodite gonad (Mello, C. C., Kramer, J. M., Stinchcomb, D. and Ambros, V. 1991. Efficient gene transfer in C. elegans: extrachromosomal maintenance and integration of transforming sequences. EMBO J. 10: 3959-3970), or by microparticle bombardment (biolistic transformation) that increases the occurrence of homologous recombination between introduced DNA and the nematode chromosomal locus (Berezikov, E., Bargmann, C. I. and Plasterk, R. H. A. 2004. Homologous gene targeting in Caenorhabditis elegans by biolistic transformation. Nuc. Acids Res. 32, No. 4 e40).

Although the invention has been described in conjunction with specific embodiments thereof it is evident that many alternatives, modifications and variations will be apparent to those skilled in the art of nematology. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A process for maintaining free-living nematodes having an increased feeding value, for use in aquaculture, comprising the steps of (a) enriching nematodes with aquaculture feed additives; and (b) preserving enriched nematodes,
wherein the step of preserving enriched nematodes includes lowering the temperature of the enriched nematodes to significantly reduce the metabolism of the enriched nematodes to the point where the metabolism of the enriched nematodes is slowed down but not suppressed to the point to dormancy, so as to maintain the nutritional value of the nematodes for feeding in aquaculture.

2. The process of claim 1, further comprising the step of: (c) culturing the nematodes.

3. The process of claim 1, further comprising the step of: (d) storing the preserved enriched nematodes.

4. The process of claim 1, wherein said free-living nematodes are selected from the species consisting of Panagrellus and Caenorhabditis elegans.

5. A process for maintaining free-living nematodes having an increased feeding value, for use in aquaculture, comprising the steps of (a) enriching nematodes with aquaculture feed additives; and (b) preserving enriched nematodes,
wherein the step of enriching nematodes includes genetic manipulation of the nematode.

6. The process of claim 5 further comprising the step of: (c) culturing the nematodes.

7. The process of claim 5 further comprising the step of: (d) storing the preserved enriched nematodes.

8. The process of claim 5, wherein said free-living nematodes are selected from the species consisting of Panagrellus and Caenorhabditis elegans.

9. An improved method of aquaculture comprising the steps of (a) enriching free-living nematodes with aquaculture feed additives; (b) preserving the enriched nematodes; (c) storing said preserved enriched nematodes; (d) rehydrating and reviving said preserved enriched nematodes; and (e) feeding rehydrated enriched nematodes to an organism being grown in the aquaculture,
wherein the step of preserving the enriched nematodes includes lowering the temperature of the enriched nematodes to significantly reduce the metabolism of the enriched nematodes to the point where the metabolism of the enriched nematodes is slowed down but not suppressed to the point to dormancy, so as to maintain the nutritional value of the nematodes for feeding in aquaculture.

10. The method of claim 9 wherein said free-living nematodes are selected from the species consisting of Panagrellus and Caenorhabditis elegans.

11. An improved method of aquaculture comprising the steps of (a) enriching free-living nematodes with aquaculture feed additives; (b) preserving enriched nematodes; (c) storing preserved enriched nematodes; (d) rehydrating and reviving said preserved enriched nematodes; and (e) feeding rehydrated enriched nematodes to an organism being grown in the aquaculture,
wherein the step of enriching nematodes includes genetic manipulation of the nematode.

12. An improved method of aquaculture comprising the steps of (a) preserving free-living nematodes; (b) storing said preserved nematodes; (c) rehydrating and reviving said stored preserved nematodes; (d) enriching nematodes with feed additives; and (e) feeding rehydrated enriched nematodes to an organism being grown in the aquaculture,
wherein the step of preserving the nematodes includes lowering the temperature of the nematodes to significantly reduce the metabolism of the nematodes to the point where the metabolism of the nematodes is slowed down but not suppressed to the point to dormancy, so as to maintain the nutritional value of the nematodes for feeding in aquaculture.

13. An improved method of aquaculture comprising the steps of (a) preserving free-living nematodes; (b) storing said preserved nematodes; (c) rehydrating and reviving said stored preserved nematodes; (d) enriching nematodes with feed additives; and (e) feeding rehydrated enriched nematodes to an organism being grown in the aquaculture,
wherein the step of enriching nematodes includes genetic manipulation of the nematode.
